(19) United States
(12) Patent Application Publication
(10) Pub. No.: US 2004/0152961 A1
(43) Pub. Date: Aug. 5, 2004

(54) Device for monitoring a patient

(76) Inventors: Sven-Erik Carlson, Herrliberg (CH); Gregor Zund, Herrliberg (CH)

Correspondence Address: Notaro & Michalos, Suite 110, 100 Dutch Hill Road, Orangeburg, NY, 10962-2100, US

(21) Appl. No.: 10/476,952

(22) PCT Filed: May 7, 2002
(86) PCT No.: PCT/CH02/00247

(30) Foreign Application Priority Data
May 7, 2001 (EP) 01110969.1
Jul. 2, 2001 (EP) 01116028.0

(51) Int. Cl. A61B 5/00

(52) U.S. Cl. 600/301; 600/322; 128/904

(57) ABSTRACT

The invention relates to a device for recording and/or monitoring medical data, particularly data pertaining to the cardiovascular condition and to the blood properties of an individual having, for example, cardiovascular disorders or diabetes. Said device comprises at least one measuring sensor (3, 3′, 23), in particular, an car sensor for detecting the cardiovascular condition of the individual (1) and comprises a logic controller for determining irregularities in the data recorded by the measuring sensor. The device also comprises a transmitting-receiving device (5) for voice and/or data in order to dial up at least one third party (9) and to transmit data thereto. Finally, the device comprises a locating system module by means of which the location of the individual is transmitted to the third party.

The present invention relates to a configuration for acquiring and/or monitoring medical data, in particular the cardiovascular status as well as blood properties of a person according to the preamble of claim 1, a method for acquiring and monitoring the cardiovascular condition of a person, in particular of a person with cardiovascular disorders as well as the use of the configuration and the method.

Through continuous monitoring of health conditions, early detection of an abnormal health status and early alerting of third persons becomes possible.

Especially in persons with cardiovascular disorders, it is eminently important that in the event of acute cardiovascular problems necessary measures can immediately be initiated, otherwise irreparable damage or even death of the person can occur within a relatively short time.

But, if appropriate, other medical disorders can also be monitored continuously, such as the blood sugar level in diabetic patients. The sugar level in the blood below or above normal can be life-threatening, such that the continuous monitoring of these values may be necessary.

Monitoring a patient in an intensive care unit, for example after cardiac infarction, with serious cardiac illness or after heart surgery can ensure that in the event problems occur, the necessary care can immediately be provided.

When transferring a patient from the intensive care unit to the hospital room permanent monitoring is already made difficult or is only conditionally possible. The patient himself can probably trigger an alarm in the event of problems, or patients connected to monitoring devices can generate an appropriate signal in the event of irregularities. Patient monitoring systems in hospitals have recently become known with which, upon the occurrence of problems, automatically alarm signals can be conducted to, for example, a supervisory person, such as a ward nurse. However, these monitor systems only function faultlessly as long as the patient is within a monitored sector. But precise position finding of the patient is not possible since the known systems are not truly portable.

For example, one such method widely used for monitoring vital parameters is acquiring the health status by means of pulsoxymetry. Pulsoxymetry, such as is for example described in WO01/41634, permits the immediate in vitro measurements of the arterial oxygen saturation by determining the color of the blood between a light source and a photodetector. In the normal case light of two different wavelengths is used, such as for example 660 nm and 940 nm. The method rests on the absorption of light in the irradiated tissue, where the light transmission is inversely proportional to the concentration of hemoglobin. During each cardiac cycle the light absorption changes cyclically: during diastole through venous blood, tissue bone and pigment, during systole through arterial blood, capillary blood, venous blood, bone and pigment.

Suitable for pulsoxymetric measurements are parts of the body such as fingers, toes, ear lobes and the like, i.e. parts where the light absorption can be visually detected.

A change of the vital health status can be demonstrated by means of pulsoxymetry. Based on the plethysmographic curve, the heart rate, respiratory frequency as well as also the oxygen saturation can be determined directly.

In particular, the cardiovascular status can be monitored by means of pulsoxymetry, and this can be carried out on healthy persons as well as also on persons who suffer from cardiovascular disorders.

As discussed above, measuring instruments for pulsoxymetry are applied especially in hospitals for monitoring patients in highly diverse fields. U.S. Pat. No. 4,685,464, WO 00/78209, WO 01/13790 and WO 01/41634 describe for example clip-like devices, which are preferably placed on fingers to make possible pulsoxymetric measurements by means of a light source and a corresponding sensor.

Instead of a sensor to be placed on a finger, U.S. Pat. No. 3,815,583 proposes a light sensor which is placed on the ear of a patient. By means of this sensor the heart rate of a patient can be measured and, upon the occurrence of irregularities or, in the event the heart beat is absent, an appropriate alarm is triggered. U.S. Pat. No. 5,910,109 similarly proposes a glucose measuring device for determining the blood sugar level in the blood. The measurement, again, takes place by means of a light source, which can be disposed on a part of the body, such as a finger or an ear, which makes superfluous the conventional wet procedure for determining blood sugar which today is still carried out by means of injection needles. However, the device proposed in U.S. Pat. No. 5,910,109 is intended for stationary application.

All of these devices have in common that a cable connection for power supply and data exchange exists between sensor and evaluation unit and that the evaluation units are relatively large and were conceptualized for stationary rather than mobile application. Therefore continuous monitoring independent of location of freely moving persons is only possible within limits.

But, it is important that for example in patients, who have been discharged from the hospital, in non-hospitalized persons, who suffer from cardiovascular disorders, in persons within a risk group, such as for example persons with a positive family history of cardiovascular diseases, or who have other risk constellations, but also for healthy persons, who prefer optimum monitoring of their health, or, for example in the case of high-performance athletes, whose health state and/or physical performance capability should be monitored, movement-independent, location-independent and continuous monitoring of the same be possible.

Specifically in persons discharged from a hospital or in risk groups the monitoring problematic is intensified. Practically the only option available is that a person in the event problems arise, can trigger an alarm, for example by actuating a button worn on the person, with which, for example, a telephone alarm can be triggered. However, in many cases the person is no longer capable of doing so and, in addition, the third party receiving the alarm does not know precisely where the person is located. This is especially the case if the person can no longer provide this information.

It is therefore a problem addressed by the present invention to propose a configuration which makes possible the monitoring of the health status of a person, which is as much as possible continuous and independent of movement and/or location.

A further problem addressed by the present invention is providing a configuration by means of which a person, in particular with cardiovascular problems or with problems of blood sugar levels, can be monitored and located at any time.

Proposed is a configuration in particular according to the terms of claim 1.

Proposed is a configuration for monitoring, which comprises at least the following components:

at least one measuring sensor on the person for acquiring medically relevant data, such as in particular data, which describe the cardiovascular function and/or contain information regarding the properties of blood or composition of the blood, which sensor comprises at least one light source which can transmit light at least at two frequencies, as well as at least one light receiver for acquiring the light penetrating through a portion of the tissue, or to determine the absorbed or reflected light,

if necessary, a logic control for the sensor to determine whether or not the measured values are within or outside a defined normal range,

a transmitting and receiving device for voice and/or data, to address, if appropriate, a third party and transmit data to this party, as well as, if appropriate and optionally,

a positioning system which makes possible the precise position finding, such as for example a GPS (Global Positioning System) module, by means of which the location is transmitted to the third party.

The measuring sensor(s) which monitor(s) the health status of the person, advantageously acquire as many relevant medical data as possible, such as for example heart rate, respiratory frequency, oxygen saturation, cardiac output, EKG data, blood pressure, blood sugar and possibly further factors, such as body temperature, etc. The sensor(s) is (are) to be placed on or in the body such that they ensure maximum freedom of movement and miminum interference with normal life. All sensors are advantageously disposed in a single sensor unit, which can be worn, for example as a wrist band, finger clip, on the ear or subcutaneously. It is understood that this sensor unit can also be disposed on any other position of the body.

The sensor(s) is (are) controlled by a logic, which checks whether or not the measured value are within or outside of the normal range defined by a physician of the person or the patient. If measured values outside of the normal range are detected, the sensor units sends by means of a wire connection or preferably a wireless connection, such as for example a so-called radio transceiver, a command to a data device, transmitting and receiving device for voice and/or data, worn by the patient, to establish automatically a connection to at least one receiver, such as for example a preprogrammed telephone number or Internet address.

This transmitting and receiving device can be a mobile telecommunication apparatus, such as for example a so-called GSM telephone (Global System for Mobile communication), a UMTS apparatus (Universal Mobile Telecommunication System), etc., which apparatus are generally conventionally employed as wireless communication means or as replacements for stationary telephone communication. In principle, any mobile telecommunication apparatus can be used, which transmits wirelessly data and/or voice information, be that via a telecommunication network or via the Internet. If necessary, with this mobile telephone an additional unit must be provided, comprising a device for wireless communication with the sensor unit as well as a control electronic for the automatic addressing of a receiver. For the wireless communication between sensor unit and transmission device, such as said GSM telephone, a data communication in the radio frequency range comes to mind, such as for example the so-called “Bluetooth” technology recently employed for local voice and data communication, which, in extremely simple manner and utilizing extremely small modules, makes possible wireless information exchange between several apparatus. This Bluetooth technology has recently also be employed with said GSM telephone apparatus, which makes disposing said additional unit superfluous.

The “Bluetooth” technology operates in the 2.4 GHz range and utilizes a relatively elaborate communication protocol. A a consequence, it has a relatively high power consumption. Since the power saving in the application defined according to the invention is very important, it can be advantageous to utilize a lower frequency and a simpler protocol specifically tailored to this purpose.

So that, as mentioned above, a receiver, such as for example a medically trained person or a physician on call in a hospital, also knows, in addition to the fact that with the person to be monitored serious health problems occurs, where the person is located the invention proposes to utilize a position finding system, such as a so-called GPS technology. Recently mobile telephones have been offered on the market, which, in addition, make possible so-called GPS (Global Positioning System) navigation. Therewith, in addition to the data characterizing the cardiovascular status, the position coordinates of the person are now transmitted to the receiver, who consequently knows immediately where the person is located. The receiver can either seek out the person himself or can, for example, summon an emergency medical service or an emergency physician in the vicinity of the person.

A further advantage when utilizing a mobile telephone for the transmission of data from patient to receiver lies therein that voice and data can be transmitted simultaneously in both directions. Specifically through the newly developed technologies such as UMTS, GPRS (General Packet Radio Service), etc. it is possible to conduct voice and data communication simultaneously from a mobile telephone to external sites. The receiver, such as for example, the family physician or a medically trained person, can attempt to communicate with the patient if the latter is conscious and able to speak. In other words, the utilization of a mobile telephone makes it possible for the receiver to take up direct contact with the person to be monitored during the transmission of the medical data. The data communication takes place directly and automatically if the data have fallen below or have exceeded a predetermined alarm limit and by the establishing of communication with the appropriate connection.

With the communication or data exchange in both directions it is additionally possible that the receiver can query data at the sensor unit, in order to be able, for example, to track the heart rate over a certain length of time. These data can be displayed, for example on a screen, such that the status of the patient is optimally represented.

According to a preferred embodiment variant it is proposed that in individual operator chips, such as the so-called Subscriber Identity Module (SIM), conventionally present in the mobile telephone, data are stored, which make it possible for the receiver or a medically trained person to view the personal medical history of the person to be monitored. For example, on such a SIM card the medical history with X-ray images and/or X-ray films and/or a list of medications can be stored, such that in the event of an emergency, the data can be transmitted to a receiver and in this way the rapid and correct response in the medical treatment is made possible. This reduces markedly the morbidity and mortality.

These so-called SIM cards are normally equipped with 32 Kbytes of storage space. Of these the files related to the mobile telephone require only approximately 10-15 Kbytes of storage space. The remaining storage space is available for other applications. The technological development moves toward making available in the future more storage space on these SIM cards and to make possible customer-specific applications and additional applications. The first cards with 64 Kbytes of storage space have recently come on the market and 128 Kbyte cards are expected to be available in 2002 at the latest. This development will continue. It must also be assumed that in the future other standardized methods, similar to the currently used SIM cards, will be developed in order to make available customer-specific data in the mobile telephone or mobile telecommunication apparatus.

Against this background it is now possible to store the medical history with X-ray images and/or X-ray films and/or medication lists or, if necessary for reason of storage space, a summary thereof of a person associated with the mobile telephone. Depending on the condition, the person associated with the SIM card of the mobile telecommunication apparatus can assign to a third party the access rights to his medical history. The data, if possible, are protected by a password. But it is also possible that the person himself sends the data to a third party. A third party can call up the data on his receiving apparatus if necessary. The data can also be sent to a third party, for example a rescue center connected to the system, as soon as the sensor unit of the measuring sensor generates an alarm.

Access to the medical history makes it possible for a third party in a medical emergency situation of the wearer of the measuring sensor to obtain a fast overview over the present medical suffering and medication application in order to initiate subsequently the correct medical measures.

On such a SIM card or a similar data storage, for example, the following information can be stored:

name and address of the person or the patient

physician treating the patient

family members to be notified

personal medical history or portions thereof

information regarding the insurance of the person.

It is essential that the stored data or the medical history always remain with the patient, and only in emergency situations are made available to a rescue service center.

By knowing additionally the precise location of the patient, based on the data available to the receiver, emergency actions can be triggered corresponding to the condition of the patient.

It is known that worldwide increasingly more persons are suffering from cardiovascular disorders. These persons fear that their cardiovascular disorders could become life threatening short-term and without prior warning. Through the configuration proposed according to the invention such patients are offered the option of automatically notifying a third party, such as for example a medically trained person, if the health conditions change such that they become life threatening or health threatening. The system proposed according to the invention improves the quality of life of the patient due to the increased sense of safety. The system decreases the response time between the occurrence of health changes or the medical treatment by:

the substantially faster arrival at the patient of emergency rescue services

the capability of assessing the health condition due to the data transmitted via telecommunication.

The system, moreover, ensures optimum utilization of material and personnel when starting out since the initial diagnosis is known and determining the position has already been completed.

The system or the configuration proposed according to the invention can contribute to reducing functional injuries in survivors and can potentially even be life-saving.

The configuration proposed according to the invention is suitable for example for monitoring persons with cardiovascular disorders or diabetics in order to generate for example an alarm at a rescue center, if the values determined by the sensor unit are outside a predetermined range or if they fall below or above alarm limits. The configuration can further be utilized for healthy persons, who consider increased safety in daily life as being desirable.

A further application is cardiovascular monitoring or blood sugar level monitoring in connection with a medical clarification. For example, in a periodic medical check such as a check-up, the physician can order said cardiovascular monitoring, under which the person must for a certain length of time carry, for example, a so-called EKG apparatus. As an alternative, and preferably, it is proposed that on this person an ear sensor, proposed according to the invention is disposed, which has high wearing comfort, with which factors meaningful regarding the person's health status can be measured and which permit simple recording. The preferably proposed ear measuring sensor will be discussed in detail in the following. But the physician can order a regular check of the blood sugar level, which is significantly simpler with the device proposed according to the invention than by means of the conventional method where a person must carry out the periodic tests by means of an injection needle.

Again, a further application is monitoring infants in order to avoid sudden infant death by generating an alarm through the measuring sensoric system received by the parents/caretakers.

Again, a further application of the configuration proposed according to the invention lies in the monitoring of athletes, in which measured values can be continuously transmitted and evaluated for the purpose of providing proof of performance. Monitoring athletes in the above sense, i.e. for monitoring the cardiovascular system is, of course, also possible.

This monitoring can also be performed within the scope of a self-check or monitoring, thereby that an athlete or, for example, a diabetic patient can periodically check a measured value “on himself”, or a signal is triggered on the person himself if there is too strong a discrepancy.

A further application of the configuration proposed according to the invention lies in the monitoring of dental patients during dental interventions to check the status of the patient.

It is understood that the above list represents only examples and is not conclusive.

According to a further preferred embodiment, the measuring sensor is

a device which can be placed on the ear, which comprises one part each to be placed at least on two sites of the ear lobe and/or the outer ear,

one part comprising a member for light emission, and

the other part comprising a light sensor for determining the light transmitted through the lobe and/or outer ear, as well as

a transmitter for the wireless transmission of the values determined by the sensor, or the evaluation data derived therefrom, to the transmitting and receiving device, such as the mobile telecommunication apparatus.

The measuring devices described within prior art are, as a rule, such which are preferably placed on a finger of a person such as for example a patient.

The disadvantage of measuring devices placed on fingers lies therein that values, such as for example the blood pressure, are different depending on whether or not a hand hangs down or, for example, is held above the head. Consequently, these are disturbance factors which possibly can yield false measuring values or which can make the evaluation of the determined measured values difficult. Disposing the measuring device on the ear lobe or the outer ear for that reason is advantageous since disturbance factors, due to different head position and movement, are significantly lower. For this reason the invention proposes disposing the measuring electronics system on an ear lobe or the outer ear and it is significant that measured data can be transmitted to a receiver without cable connection to make possible movement and location independence of the person to be monitored. The measuring of the medical data preferably takes place by means of pulsoxymetry or by means of the so-called live-check method, in particular for acquiring the blood sugar content.

It is understood that such a transmitting device, due to its placement on an ear or in the region of an ear lobe or the external ear, must be formed such that it is of minimum size. For this reason it is proposed according to the invention that the transmission of the data measured by the sensor or data derived by an evaluation device takes place by means of radio frequency technology.

The configuration proposed according to the invention preferably comprises a securing device on the ear, such as for example a bow, a clamp, clip, a part extending through the ear or an adhesion connection. It is essential that the measuring sensoric system is disposed stably on the ear lobe or the external ear, in order to make possible a continuously constant measurement, and to minimize the disturbance factors as much as possible. The configuration further comprises the measuring sensoric system on the ear lobe, such as described in the introduction, as well as, if appropriate, an electronics system for the signal processing and signal analysis. Lastly, the configuration comprises a battery, possibly with solar cells, for the power supply as well as a transmitter in the radio frequency range and possibly receivers for the communication with an external apparatus for the purpose of data transmission. The external apparatus can be either directly a receiver which assumes authority for monitoring the health status of the person, or the above described transmitting and receiving device for voice and/or data, which establishes a connection to an external receiving center, such as for example an alarm center.

It is conceivable to supplement the measuring sensor unit on the ear lobe to make possible additional continuous measurements, such as for example determining or calculating the partial CO₂ pressure pCO₂ (degree of CO₂ saturation in blood, CO₂ pressure in arterial blood), blood pressure as well as also the blood sugar content, hemodilution, hematocrit and hemoglobin.

The evaluation of the sensor signals as well as the curves resulting therefrom and the further conduction of the results takes place by means of a signal processing and signal analysis device and a transmitting apparatus, which is placed for example by means of ear bows behind the external ear.

As already mentioned, for the wireless transmission of the data preferably data communication in the radio frequency range is utilized, which, in extremely simple manner and utilizing extremely small modules, makes possible the wireless information exchange between several apparatus. Voice and data communication can take place, for example, by means of the so-called “Bluetooth” technology, or by any other radio frequency and transmission protocol.

Through the measurements on the ear lobe very good measurement results with few disturbances can be expected, since these measurements have low sensitivity to body movements, and only entail a small standard error (distance from the heart to the ear lobe).

In the following the invention will be explained in further detail by example and with reference to the attached Figures. Therein depict:

FIG. 1 the principle and operational function of the present invention in conjunction with a schematic diagram,

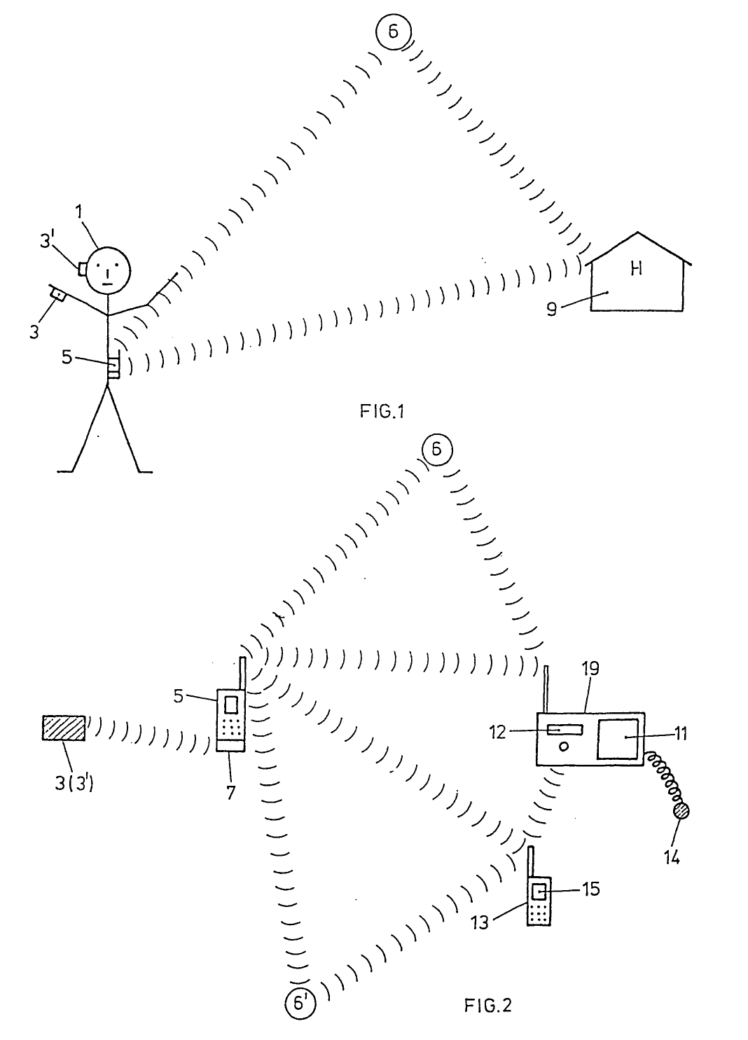

FIG. 2 in conjunction with a further schematic representation the individual elements and the operational principle of the present invention,

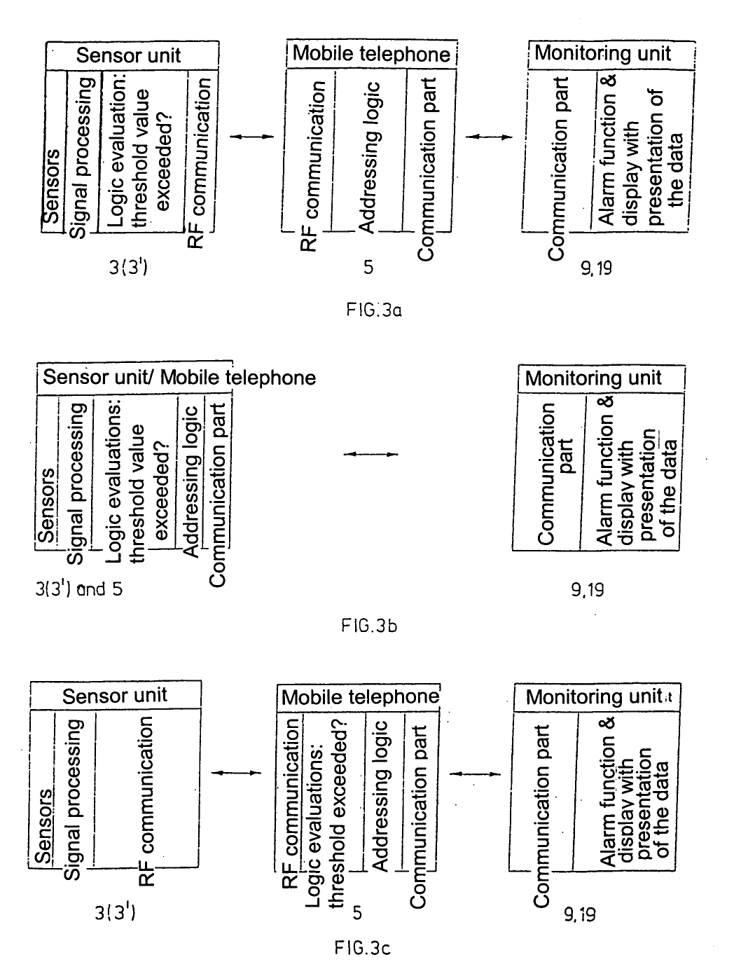

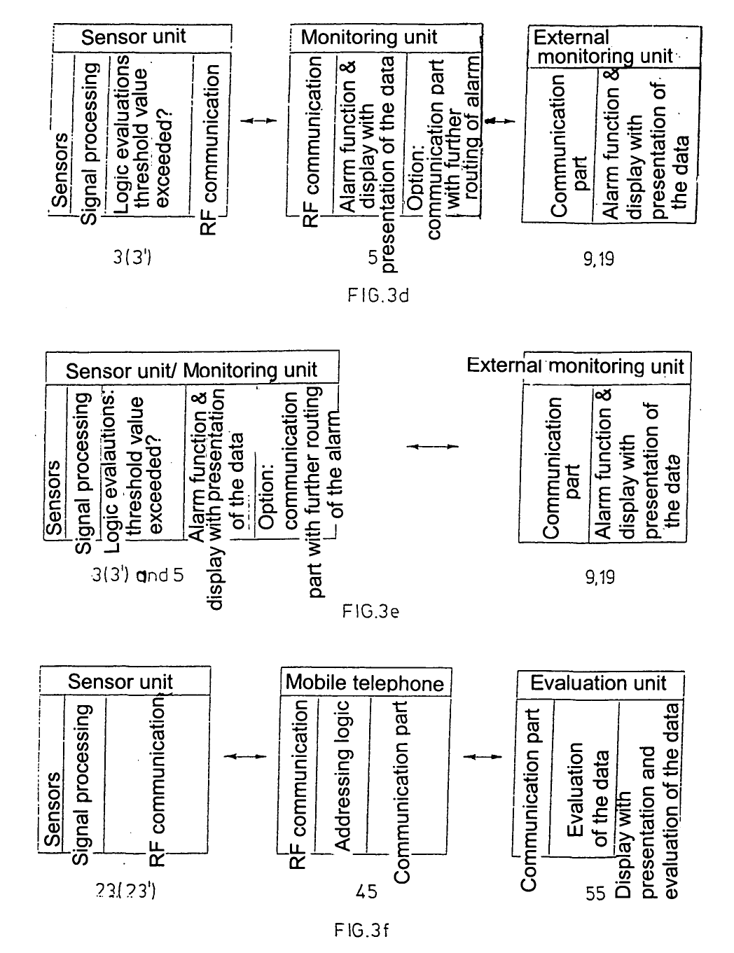

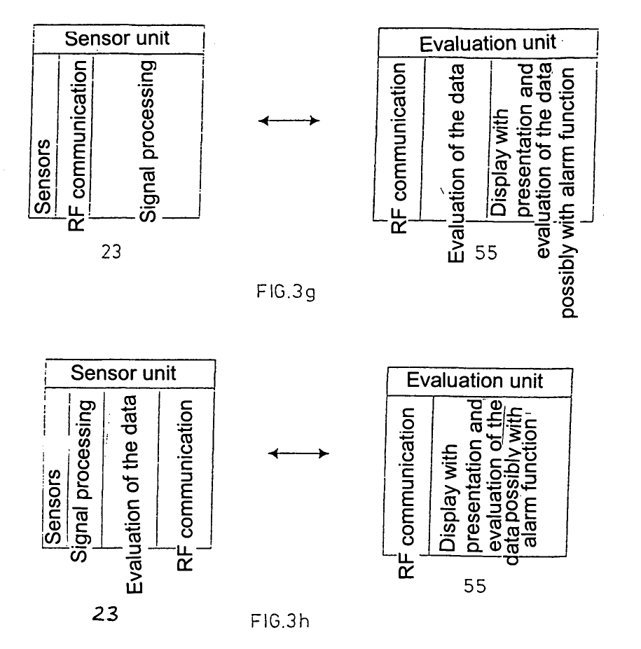

FIGS. 3a to 3h possible development variants of the configuration according to the invention in conjunction with schematic representations of the invention,

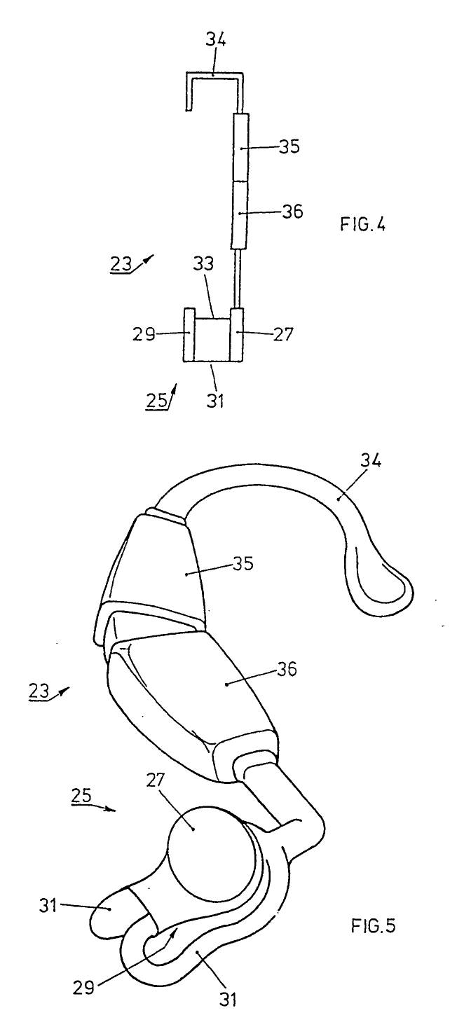

FIG. 4 schematically a configuration according to the invention for pulsoxymetry measurements on an ear,

FIG. 5 in perspective a possible development of a configuration according to the invention on an ear,

FIG. 5a a detail from FIG. 5,

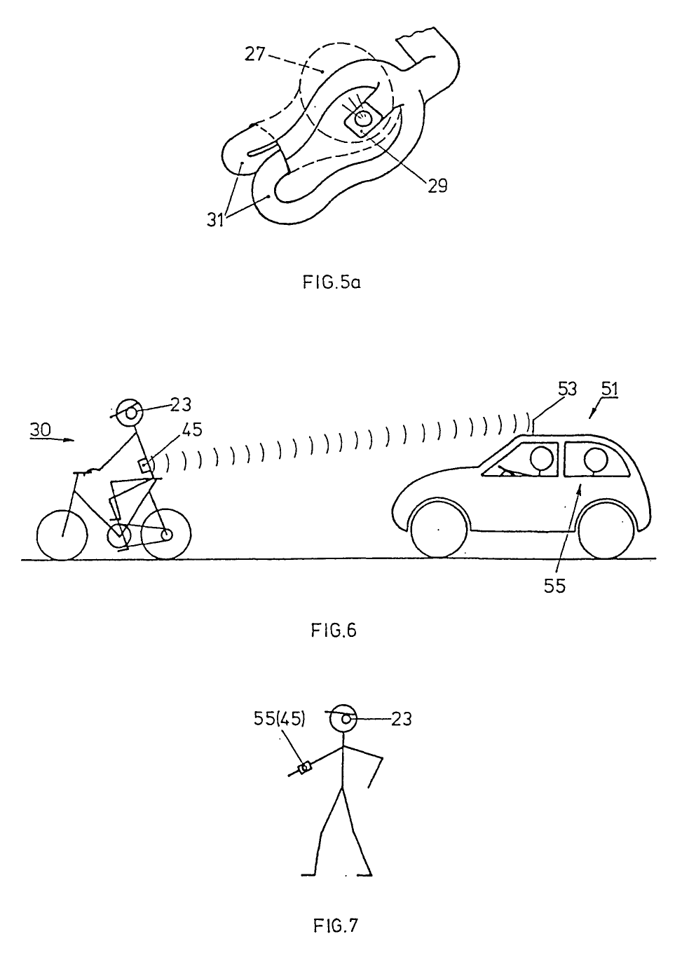

FIG. 6 again, in conjunction with a schematic representation the monitoring of the health status of an athlete by means of the configuration defined according to the invention, and

FIG. 7 schematically the self-monitoring or -check by a person.

FIG. 1 depicts the operational function of the present invention in conjunction with a schematic representation.

A person 1 is a person with cardiovascular disorders. This person can be a patient, who is under medical care, or a person who has recently been discharged from a hospital, to which he had been admitted, for example due to cardiac infarction, or in which hospital he underwent heart surgery. What is significant is that it is suspected that the person 1 may suddenly experience heart problems, which are a serious threat for the person 1. For that reason it is important that the person 1 is continuously under a physician's control, i.e. that the health status of person 1 can be continuously monitored.

This takes place by means of a sensor unit 3 or 3′, which can comprise one or several sensors, by means of which, for example heart rate, respiratory frequency, oxygen saturation, blood pressure, cardiac output, body temperature and, if necessary, additional factors relevant to heath, such as blood sugar level, can be monitored. The sensor unit can be disposed, for example, like a wrist band or a finger clip, as denoted in FIG. 1 by the reference number 3, or on an ear, as denoted in FIG. 1 by the reference number 3′. Further disposed in the sensor unit 3 is a logic control, which checks continuously whether or not the measured values are within or outside a normal range as defined by a physician of the patient. If measured values outside the normal range are detected, the sensor unit outputs by means of a wire connection or by means of a wireless connection, such as preferably a so-called radio transceiver, a command signal to a mobile telephone 5, which is also on the patient. Due to this signal, in the mobile telephone, which can be for example a so-called GSM telephone (Global System for Mobile communication), a selection pulse is triggered by means of which one or several receivers are addressed. The receiver can be for example an emergency rescue center 9, which is operated for example by a medically trained person. Upon the establishment of a connection by the medically trained person, via the connection from the mobile telephone 5 to the terminal in the hospital 9, such as a telephone station or an Internet connection, the data measured by the measurement unit are transmitted to this medically trained person, such that the medical person, based on these data and the identification of the patient, which is also made possible through the mobile telephone 5, can immediately prepare an assessment of the health status and initiate appropriate measures.

It may be important that the medically trained person knows the position coordinates of patient 1, so that he knows the location of the patient. This can be determined, for example, by means of the so-called and already widely established GPS system (Global Positioning System), by transmitting from the mobile telephone 5 additionally to the data transmission, also the position coordinates via satellite 6 by means of said GPS system. It is understood, that other location configurations are also conceivable, such as for example navigation by means of the GSM network, such as for example the Location Based Service (LSB), which is offered by the Swiss telecommunication company Swisscom.

In the rescue center the decision can now be made whether or not a team of the hospital or an external site should be summoned, in order to provide the patient with the necessary help.

The present invention or the functional principle will be explained in further detail in conjunction with the scheme of FIG. 2.

As already stated, data measured by the sensor unit 3 or 3′, in the event they differ from a predetermined measuring range, are transmitted, for example wirelessly, to a mobile telephone unit 5. For the communication between sensor unit 3 or 3′ and the mobile telephone 5 a wire connection can exist as well as also a wireless one, such as for example by means of infrared, thereby that on the sensor unit as well as the mobile telephone an infrared interface for data transfer is provided, and further suitable is in particular the data transfer in the radio wave range, such as for example by means of the so-called “Bluetooth” technology. This technology ensures the information exchange between apparatus without the use of any cable connections. This “Bluetooth” technology has recently been used for example in connection with so-called notebooks or laptop personal computers, thereby that these mobile personal computers are connected at any time wirelessly within a certain range with a central unit, and consequently the wireless data communication is possible at any time. But also in the field of mobile telephones the utilization of said “Bluetooth” technology is being proposed. It is known that the “Bluetooth” technology operates in the 2.4 GigaHertz range and utilizes a complicated communication protocol. This leads to a relatively high power consumption. Since power saving in the applications proposed according to the invention is important, it could be advantageous to utilize a lower frequency and to employ a simpler, specially tailored protocol. Depending on the manner in which the data communication between sensor unit 3 and/or 3′ and mobile telephone 5 takes place, on the latter an additional unit 7 must be disposed or installed, which comprises a device for the wireless communication with the sensor unit as well as a control electronics.

In the case of said “Bluetooth” technology, which lately has also been integrated in mobile telephones, the necessity of disposing said additional unit 7 becomes superfluous.

In the event the measured data deviate from a predetermined, defined range the mobile telephone 5 addresses automatically a receiver, such as for example a telecommunication apparatus 19, which is connected to a data acquisition and evaluation unit. On it on displays 11 and/or 12 the data measured by the sensor unit 3 or 3′ are reproduced such that a person on call at the receiver unit 19 can immediately carry out an assessment of the status regarding the health of the patient. By means of the position coordinates of the location of the patient or the mobile telephone 5, transmitted via a satellite 6 of the GPS system, the person on call can furthermore immediately determine, for example on a screen 11, the location of the patient. Consequently, upon the occurrence of health problems of the patient the medically trained person on call can virtually without delay initiate the necessary measures in order to help the patient. In addition, it is possible, for example by means of telephone 14 to establish voice contact, since by using the mobile telephone unit 5 a simultaneous voice and data communication is possible. If the patient is responsive, the medically trained person can for example gather information from the patient about his health or about his impressions of the situation.

But it is also possible that the medically trained person receives by transmission from the storage medium, which is disposed in or on the mobile telecommunication device, such as the mobile telephone 5, data automatically together with the data measured by the sensor, such as for example the medical history of the patient, or that he can record it himself. It is known that with each mobile telecommunication device of a person or a group of persons through an identification chip, such as a so-called SIM card (Subscriber Identity Module). On this module the medical history of the person to be monitored can be stored, or additionally, data important to the medically trained person, such as name and address of the patient, family physician, family members to be notified, data regarding medication applications, medical measures already taken, etc. This information can additionally critically affect the necessary measures to be taken.

From the rescue center a further mobile telephone 13 can also be informed, which is worn, for example, by the physician treating the patient. On a display 15 of the mobile telephone 13 the data measured by the sensor unit 3 or 3′, or an abbreviated version thereof, can be read which can be transferred from the rescue center further to the mobile telephone. The treating physician wearing the mobile telephone 13 can, in turn, enter into a voice dialog with the patient. It is understood that it is also possible that the data transfer is carried out from the patient directly to the mobile telephone 13 of the treating physician, and the treating physician can also, if necessary, determine the location of the patient, thereby that the coordinates are transmitted to him via satellite 6′ by means of GPS. But as a rule, the contact or the data transmission to a rescue center is obligatory, and the message to the treating physician depends on the circumstances.

But, with the monitoring system proposed according to the invention or with the configuration it is also possible that, for example, the family physician from time to time calls up data at the sensor unit 3 or 3′ via the data communication chain, in order to obtain in this way an impression of the health status of a patient.

The monitoring unit proposed according to the invention is also suitable for self or personal checking in order to acquire data relating to sports medicine or to be able to call them up at any time. Known are, for example, measuring devices worn on a chest belt, which are provided to acquire and reproduce the heart rate, blood pressure as well as other data, such as running distance, duration of the sports activity, etc.

It is understood that the two schematic diagrams depicted in FIGS. 1 and 2, are only examples to explain the present invention in further detail. The elements selected in the diagrams as well as the described transmission technologies depend on the currently customarily employed technologies and capabilities. In particular, mobile telephones with integrated GPS system have only recently been available on the market and are only offered by a few manufacturers, such as for example the Finnish company Benefon OY. But it must also be assumed that such devices will shortly also be offered by other manufacturers. With respect to the “Bluetooth” technology, it must be added that this technology is only used in a few apparatus and systems. But this technology or related technologies will in the future also decisively influence especially the area of data processing and data communication, such that these technologies can of course be applied accordingly in connection with the present invention. With respect to the measuring sensor as well as logic control, different formations are also conceivable. For example, the measuring sensors can be disposed in a ring to be worn on the finger and the evaluation electronic or logic control in a wrist watch, in which case the data transmission can take place via infrared interface or radio waves. But measuring sensor and evaluation electronics as well as logic control can all be disposed in a wrist watch or generally in a wrist band. Lastly, sensor as well as evaluation electronics and logic control can be disposed at any other suitable location on the body and utilizing a suitable carrier.

In FIGS. 3a to 3h possible implementations of configurations are shown in conjunction with diagrams with reference to the possible applications of the monitoring configurations.

FIG. 3a shows in conjunction with three units 3 (3′), 5 and 9, 19 a possible layout of a monitoring configuration for the medical monitoring of a patient. The sensor unit 3 or 3′ comprises the following components: sensors, signal processing member, logic evaluation unit for determining if the acquired measured data fall below or exceed programmed threshold values, as well as a communication member for the data communication in the radio frequency range.

The mobile data communication unit 5, in turn, comprises a member for the data exchange in the radio frequency range, an addressing logic for addressing an external third party as well as a communication component.

Lastly, the diagram according to FIG. 3a includes a monitoring unit 9 or 19, with again a communication component as well as a display for presenting the data measured and, if appropriate, utilized by the sensor unit.

According to a further implementation variant in FIG. 3b, it is of course possible to combine the sensor unit 3 (3′) as well as the mobile data communication unit 5 into one single component.

FIG. 3c shows a further variant of a monitoring configuration in which the logic control is not provided in the sensor unit 3 (3′), but rather in the mobile data communication unit 5.

FIG. 3d shows another application option in that here the monitoring configuration is used for the observation or monitoring of an infant. The sensor unit 3 (3′) is structured analogously to that of FIG. 3a. But, in comparison in the monitoring unit 5 a display is already provided or a display for the representation of the data acquired in sensor unit 3 (3′). If these fall below or exceed a threshold value, an alarm can already be provided in the monitoring unit. It is lastly possible to provide also on the monitoring unit 5 a communication component, in order to transmit the data further to an external monitoring unit 9, 19 or to an alarm unit, where an external alarm can be triggered.

FIG. 3e shows a further variant of infant monitoring, in which the logic control is not provided in the sensor unit 3 (3′), but combined with the sensor in the monitoring unit 5.

In FIG. 3f a monitoring configuration for a so-called third party sports monitoring is shown schematically. In the configuration according to FIG. 3f the sensor unit 23 (23′) comprises only one or several sensors, as well as a signal processing member from which the data are transmitted by means of radio frequency to a data transmission unit, such as for example a mobile telephone 45. From this mobile telephone 45 the data are subsequently transmitted by means of a communication component to a monitoring or evaluation unit 55.

FIG. 3g shows in conjunction with a diagram a simple sports monitoring unit, where data measured and evaluated in a sensor unit 23 are transmitted by means of RF communication to an evaluation unit 55, such as for example to a wrist watch worn on an arm. On this evaluation unit 55 a display is provided, on which the data or their evaluation can be displayed in different ways, including the alarm output if the values fall below or exceed programmed threshold values. Analogously, in this way diabetics can continuously keep their blood sugar values in check.

FIG. 3h lastly shows a further variant of a sports monitoring unit or diabetic monitoring unit, in which the logic control is not provided in the sensor unit 23, but rather in the evaluation unit 55.

Accordingly FIG. 4 shows in simplified schematic form a sensor unit according to the invention provided to be attached on the ear of the person to be monitored and to acquire the medical data by means of pulsoxymetry.

A measuring sensor unit 23 comprises the measuring sensor 25 proper, comprised of a light source 29 and a photodetector 27, which are disposed each on one side of an ear lobe, which are connected, for example, via a bow-like connection 31 with one another. To fix the two elements 27 and 29 on the ear lobe it can be advantageous to provide additionally pin-like extensions 33 extending through the ear lobe such that the measuring sensor is disposed on the ear lobe immovably and fixed in position. It is understood that this position securement can also be attained through the use of a clip, a clamp, by adhesion of the elements 27 and 29 on the ear, etc.

In an ear bow 34, which extends at least partially about the external ear, are further provided a transmitter/receiver 36, as well as a battery 35. In the transmitting/receiving member 36, further a data processing unit can be provided in which the data determined by the measuring sensor 27 can be processed or evaluated. Lastly, it is also possible to enter into this data processing unit reference values or value ranges for the factors to be measured, such as respiratory frequency, oxygen saturation, heart rate, etc. and if the values fall below or exceed the specified ranges, an appropriate alarm signal is generated. The transmitting/receiving member is a unit operating in the radio frequency range, i.e. the transmission of the data takes place in the radio frequency range.

In FIG. 5 is depicted a more concrete embodiment variant of the configuration according to FIG. 4 in perspective, provided to be disposed about one ear of the person to be monitored. Again, the configuration 23 comprises the measuring sensor unit 25, comprising a light source 29 (not shown) as well as the measuring sensor 27. For fixing and connecting the two elements 27 and 29 further a positioning device 31 is provided, such as for example a clip bow 31. Disposed on the ear bow 34 are again the battery unit 35 as well as the RF transmitting/receiving member and data processing unit 36. The operational function of the measuring sensoric system rests on the light absorption in the irradiated tissue in the ear lobe, and the light transmission is inversely proportional to the concentration of hemoglobin. The light absorption varies cyclically during each heart cycle. Due to the rapid absorption time and the reliability of the measurements, an ear lobe is best suited for pulsoxymetric measurements. The measurement of the arterial oxygen saturation is obtained based on the determination of the color of the blood between the light source and the photodetector 27.

FIG. 5a shows a detail from FIG. 5, in which the representation of the measuring sensor 27 has largely been omitted. By omitting the sensor, the light source 29, not visible in FIG. 4, becomes visible.

In FIG. 6 is shown schematically a further application of the manner in which the data acquired or evaluated by the measuring configuration 23 can be transmitted to a receiver or be evaluated by it.

The issue in FIG. 6 is the monitoring of the health status or the acquisition of medical data of a bicycle rider 30, in order to determine, for example, the performance of the bicycle rider, to optimize training methods, to investigate the riding style optimal for the bicycle rider, to determine generally medical data of active people, etc.

Again, the measuring configuration 23 proposed according to the invention is fastened on the ear of the bicycle rider 30, who is taking a bicycle ride. Again, the values measured or determined by the measuring sensor are transmitted to a wireless transmitting device 45, with this data transmission from measuring sensor to the data transmission unit 45 also taking place wirelessly in the radio frequency range, for example by means of the so-called “Bluetooth” technology. From the data transmitting device 45 the data are wirelessly conveyed for example to a receiving antenna 53 on an escort vehicle 51, in which the data are continuously monitored by a competent person 55. This person can be a medically trained person, a trainer or even simply an acquaintance of the bicyclist 30.

It is understood that it is not absolutely necessary that a person is present in the escort vehicle 51, since the wirelessly transmitted data can also be recorded or stored, in order to be evaluated later.

As long as the bicycle rider is within viewing range of the escort vehicle, the position of the bicycle rider is, of course, known to the medically trained person or the trainer. But precisely during sports events the situation often arises that escort vehicles and bicycle riders are positioned relatively far apart, which is why in the case of critical data measured on the bicyclist, his position is known at all times to the person in the escort vehicle. For this reason, it is again of advantage if, in addition to the transmitted medical data, position data are also transmitted to the escort vehicle, for example by means of a so-called GPS device, as already described with reference to FIGS. 1 and 2.

But, it is understood that it is also possible that the receiver is not located in an escort vehicle, but is stationary for example in a training center, where he can continuously monitor the data acquired by the measuring configuration 23 at the bicyclist. By additionally conveying the coordinates by means of the GPS device, the location of the bicyclist is also known at any time, for which reason, in the event an intervention becomes necessary with the bicyclist, the trained person or the trainer can initiate the necessary measures. By knowing the position data, an auxiliary trainer or an escort person in the vicinity can, for example, be summoned or be instructed regarding potential measures to be taken.

FIG. 7, lastly, shows schematically the possibility of using the configuration available according to the invention for the self-check of an athlete or for example of a diabetic. By means of the ear sensor 23 either the cardiovascular status or pulse, blood pressure and the like can be continuously measured, which values are important for an athlete. These values are wirelessly transmitted from sensor 23 to an evaluation or display configuration 55, where the athlete can read these values continuously or periodically. Analogously, it is possible that a diabetic can read periodically the blood sugar level on display 55, or that the display 55 indicates when the normal value has been fallen below or exceeded, if such a state is measured by the sensor 23 on the ear of the diabetic patient. It is, of course, possible that in addition a data transmission unit 45 is provided on display 55, in order to transfer the data measured by sensor 23 to an external site. This self-monitoring has the great advantage in the case of diabetics, that they are informed in time if self-medication has become necessary, such as for example the self-administration of insulin. The data transmission lastly, from sensor 23 to display 55, again, takes place wirelessly.

It is understood that the various situations, depicted in FIGS. 1, 2, 3, 6 and 7 are only examples, which are suitable to explain the present invention in further detail.

The configuration proposed according to the invention can be employed in any other situation in which the health status of a person must be monitored or where medical data of a person must be acquired. As stated above, it may be advantageous to dispose the measuring sensoric system on an ear. For example, the measuring sensoric system on the ear can be integrated into an object of daily use, such as for example into a hearing aid or jewelry for the ear.

The present invention is not limited to the situations listed in connection with the two FIGS. 1 to 7, measuring sensors, communication devices, technologies and embodiment example of the individual modules, but rather comprises, especially with respect to technologies, also those which are only in the developing stage and not yet available on the market. The present invention in particular is not limited to the described applications of use. For example, as a further conceivable application the configuration proposed according to the invention is also suitable for monitoring babies, and it is unfortunate that reference must be made in this connection to the repeatedly occurring so-called “sudden infant death”.

1. Configuration for acquiring and/or monitoring medical data, in particular of the cardiovascular status, blood properties, etc., characterized by
at least one measuring sensor (3,3′, 23) for acquiring the medical data, such as cardiovascular status, etc. of a person (1), comprising at least one light source, which can emit light at least at two frequencies, as well as at least one light receiver for determining the light transmitted through a tissue portion of the person,
if appropriate, a logic control for detecting possible irregularities of the data acquired through the measuring sensor, and
a transmitting and receiving device (5, 25, 35, 45) for voice and/or data, in order to address, if necessary, at least one third party (9, 13, 19) and to transmit data to it.

2. Configuration, in particular as claimed in claim 1, characterized in that further a position finding system module is provided, by means of which the location of the person is conveyed to the third party.

3. Configuration, in particular as claimed in claim 1 or 2, characterized in that one or several measuring sensors are provided for acquiring as many relevant medical data as possible, such as heart rate, respiratory frequency, oxygen saturation of the blood, blood pressure, cardiac output, EKG data, blood sugar level and/or body temperature.

4. Configuration, in particular as claimed in one of claims 1 or 3, characterized in that as the transmitting and receiving unit (5) serves a telecommunication apparatus such as a mobile telephone, which comprises as an additional module or as an integrated structural element an automatically triggerable addressing member, which can be triggered in response to a signal of the logic control.

5. Configuration, in particular as claimed in one of claims 1 to 4, characterized in that on the transmitting and receiving unit (5) a communication and control electronics system is disposed or integrated, which is connected with the addressing member such that one or several preprogrammed telephone numbers and/or Internet addresses are addressed, and that from the transmitting unit, in addition to measured data, also position coordinates such as GPS (Global Positioning System) coordinates are conveyed to the third party.

6. Configuration, in particular as claimed in one of claims 1 to 5, characterized in that for conveying the data from the measuring sensor (3,3′, 23) or the logic control to the transmission unit (5) data communication in the radio wave range, such as for example so-called “Bluetooth” technology components or components with another transmitting frequency and/or protocol, are employed.

7. Configuration as claimed in one of claims 1 to 6, characterized in that at the third party a device (11, 12, 15) is provided, on which the data acquired by the measuring sensor can be displayed or visualized as well as the location of the person to be monitored.

8. Configuration as claimed in one of claims 1 to 7, characterized in that the transmitting and receiving device as the device at the third party are such that simultaneously data and voice communication in both directions is possible in order to make possible voice communication between the person and the receiver, even during data transmissions.

9. Configuration as claimed in one of claims 1 to 8, characterized in that at the transmitting and receiving device a memory module is provided, in which data are stored which relate to the person to be monitored, which data comprise, for example, selected from the following list:
at least partially the medical history with, if appropriate, X-ray images and/or X-ray films and/or lists of medication of the person
name and address of the person
information regarding the treating physician or regarding the treating medically trained persons
family members to be notified
information regarding the insurance protection.

10. Configuration as claimed in claim 9, characterized in that the memory module is a so-called SIM card (Subscriber Identity Module), which card is disposed in the transmitting and receiving device, in order to associate it with the person.

11. Configuration, in particular as claimed in one of claims 1 to 10, characterized in that the measuring sensor comprises
an arrangement (23) to be placed on an ear, which comprises one part each which can be placed at least on one place of the ear lobe and/or the external ear, wherein
one part comprises a member (29) for light emission, and
the other part a light sensor or a light receiver (27), for determining the light transmitted through the lobe or the external ear, and wherein
one transmitter (36) is provided for the wireless transmission of the values determined by the sensor (27), or evaluation data derived therefrom, to the transmitting and receiving device (25).

12. Configuration as claimed in claim 11, characterized in that the arrangement comprises an electronic (36) or a signal processing and signal analysis device for the analysis or evaluation of the values determined by the sensor (27).

13. Configuration as claimed in one of claims 11 or 12, characterized in that the arrangement comprises a battery (35) possibly with solar cells for the power supply.

14. Configuration as claimed in one of claims 11 to 13, characterized in that an electronic for analysis or evaluation of the determined values is provided, further comprising the logic control for the determination of irregularities of the data acquired by the measuring sensor (27).

15. Configuration as claimed in one of claims 1 to 14, characterized in that the measuring sensor is disposed such that it is integrated in a hearing aid.

16. Configuration as claimed in one of claims 1 to 14, characterized in that the measuring sensor is disposed integrated in ear jewelry or drop earrings or a so-called mobile telephone hands-free device (wireless hands-free device).

17. Method for acquiring and/or monitoring medical data, in particular of the cardiovascular status and/or the blood sugar level of a person by means of a configuration, in particular as claimed in one of claims 1 to 16, characterized in that
by means of at least one measuring sensor (3) on the person (1) the medical, in particular the cardiovascular status, is monitored,
if appropriate, by means of a logic control irregularities of the acquired data are determined,
at least in the event of irregularities, by means of a transmitting and receiving device for voice and/or data (5), if necessary, a third party is addressed and data are transmitted, as well,
by means of a position finding or navigation system, such as GPS (Global Positioning System), the position of the person is conveyed to the third party.

18. Method as claimed in particular in claim 17, characterized in that the transmission of the data from the measuring sensor to the transmitting and receiving device takes place by means of radio waves, such as for example in the so-called “Bluetooth” frequency range or with another frequency or with another protocol.

19. Method as claimed in particular in one of claims 17 or 18, characterized in that as the transmitting and receiving device (5) a GSM apparatus (Global System for Mobile communication), a GPRS apparatus (General Packet Radio Service), a UMTS apparatus (Universal Mobile Telecommunication System), etc. is employed, which, due to a signal by the logic control automatically addresses at least one third party and conveys data.

20. Method as claimed in particular in one of claims 17 to 19, characterized in that the simultaneous data and voice communication between transmitting and receiving device (5) and the third party is possible in both directions, in order for the third party to be able to establish contact with the person, or, if appropriate, can read data from the measuring sensor on the person or can affect the measuring sensor or other devices at the location of the patient.

21. Method as claimed in particular in one of claims 17 to 20, characterized in that the third party can call up at least partially the medical history of the person to be monitored from a memory module at the transmitting and receiving device (5) and, if necessary, by means of a password, as well as, if necessary, further information such as data regarding insurance protection, treating physician, family members to be notified, etc.

22. Method as claimed in one of claims 17 to 21, characterized in that the acquisition of the health status or the medical data takes place by means of pulsoxymetry, i.e. noninvasively optically by measuring the O₂ saturation, preferably on the ear lobe or on the external ear, in that light is emitted from a member for light emission (29) in at least two different wavelengths through the ear lobe or the external ear, this light is acquired by a photodetector (27) by measuring the light transmitted through the irradiated tissue in the ear lobe, the values measured by the photodetector (27) are transmitted to a sensor and, if appropriate to an evaluation electronic (36), which is also disposed in the proximity of the ear, and is transmitted from the transmitter (16) wirelessly in the radio frequency range to the transmitting and receiving device.

23. Use of the configuration as claimed in one of claims 1 to 16 for monitoring a person with cardiovascular disorders.

24. Use of the configuration as claimed in one of claims 1 to 16 for acquiring data relating to sports medicine, if appropriate by the person practicing the sport himself.

25. Use of the configuration as claimed in one of claims 1 to 16 for monitoring the health of persons with a risk constellation for cardiovascular diseases.

26. Use of the configuration as claimed in one of claims 1 to 16, for monitoring the blood sugar level in diabetics, if appropriate by the diabetic patient himself.

27. Use of the configuration as claimed in one of claims 1 to 16, for monitoring infants or babies.

28. Use of the configuration as claimed in one of claims 1 to 16 for the medical monitoring of patients in a dentist's office, in particular during dental operations of persons with medical disorders.
